(19) United States
(12) Patent Application Publication
(10) Pub. No.: US 2026/0174353 A1
(43) Pub. Date: Jun. 25, 2026

(54) Method for Determining a Respiration Rate of a Patient and Implantable Medical Device

(71) Applicant: BIOTRONIK SE & Co. KG, Berlin (DE)

(72) Inventors: Swetha VENNELAGANTI, Apex, NC (US); Ravi Kiran Kondama REDDY, Portland, OR (US); R. Hollis WHITTINGTON, Portland, OR (US)

(73) Assignee: BIOTRONIK SE & Co. KG, Berlin (DE)

(21) Appl. No.: 19/127,228

(22) PCT Filed: Oct. 6, 2023
(86) PCT No.: PCT/EP2023/077692

§ 371 Date: May 5, 2025

Related U.S. Application Data
(60) Provisional application No. 63/424,217, filed on Nov. 10, 2022.

(30) Foreign Application Priority Data
Nov. 29, 2022 (EP) 22210121.4

(51) Int. Cl. A61B 5/08; A61B 5/00; A61B 5/113

(52) U.S. Cl. CPC A61B 5/0816; A61B 5/113; A61B 5/6846; A61B 2562/0219

(57) ABSTRACT

A computer-implemented method for determining a respiration rate of a patient comprising the steps of providing a impedance and/or accelerometer signal captured by an implantable medical device, detecting a respiration of the patient once the impedance and/or accelerometer signal crosses a sensing threshold, detecting a further respiration of the patient once the impedance and/or accelerometer signal crosses the adjusted sensing threshold, and determining the respiration rate of the patient by calculating a time interval between the signal crossing the sensing threshold and the signal crossing the adjusted sensing threshold. An implantable medical device, in particular implantable cardiac monitor, for determining a respiration rate of a patient is also provided.

### CROSS-REFERENCE TO RELATED APPLICATIONS

This application is the United States National Phase under 35 U.S.C. § 371 of PCT International Patent Application No. PCT/EP2023/077692, filed on Oct. 6, 2023, which claims the benefit of European Patent Application No. 22210121.8, filed on Nov. 29, 2022, and U.S. Provisional Patent Application No. 63/424,217, filed on Nov. 10, 2022, the disclosures of which are hereby incorporated by reference herein in their entireties.

### TECHNICAL FIELD

The invention relates to a computer-implemented method for determining a respiration rate of a patient.

Furthermore, the invention relates to an implantable medical device, in particular an implantable cardiac monitor, for determining a respiration rate of a patient.

### BACKGROUND

Respiration rate is one of the vital signs that is often overlooked but is an important indicator of the patient's overall wellbeing. In patients with cardiac disease, respiration rate at rest is an elementary measure of cardiovascular function.

Respiration rate has been demonstrated to be sensitive to various pathological conditions like adverse cardiac events, pneumonia, and clinical deterioration and stressors due to physical exertion, exercise induced fatigue, emotional stress, heat, cold, etc.

Respiration rate monitoring is valuable in detecting early signs of heart failure. Long term monitoring of respiration rate can help with detection of dyspnea and shallow breathing, which are presented as initial manifestations of heart failure.

Such risks and disease management can potentially be mitigated with a device that can periodically monitor a patient's respiration rate over a long period either from the surface of the body or from within the body.

The present disclosure is directed toward overcoming one or more of the above-mentioned problems, though not necessarily limited to embodiments that do.

### SUMMARY

It is therefore an object of the present invention to provide an improved method for determining a respiration rate of a patient and a corresponding implantable medical device.

The object is solved by a computer-implemented method for determining a respiration rate of a patient having the features of claim 1.

In addition, the object is solved by an implantable medical device, in particular implantable cardiac monitor, for determining a respiration rate of a patient having the features of claim 13.

Further developments and advantageous embodiments are defined in the dependent claims.

The present invention provides a computer-implemented method for determining a respiration rate of a patient.

The method comprises providing an (filtered or unfiltered) impedance and/or accelerometer signal captured by an implantable medical device and detecting a respiration of the patient once the impedance and/or accelerometer signal crosses a sensing threshold.

Furthermore, the method comprises starting a detection hold-off period, starting a peak detection window during which, an amplitude of the impedance and/or accelerometer signal is tracked and after expiry of the detection hold-off period, setting an adjusted sensing threshold based on a measured signal peak.

The method moreover comprises detecting a further respiration of the patient once the impedance and/or accelerometer signal crosses the adjusted sensing threshold and determining the respiration rate of the patient by calculating a time interval between the signal crossing the sensing threshold and the signal crossing the adjusted sensing threshold.

The present invention further provides an implantable medical device, in particular implantable cardiac monitor, for determining a respiration rate of a patient. The implantable medical device comprises means for capturing an impedance and/or accelerometer signal, means for filtering the captured impedance and/or accelerometer signal and a control unit for detecting a respiration of the patient once the (e.g. filtered) impedance and/or accelerometer signal crosses a sensing threshold, said control unit being configured to start a detection hold-off period, start a peak detection window during which, an amplitude of the (e.g. filtered) impedance and/or accelerometer signal is tracked, after expiry of the detection hold-off period, set an adjusted, second sensing threshold based on a measured signal peak, and detect a further respiration of the patient once the (e.g. filtered) impedance and/or accelerometer signal crosses the adjusted, sensing threshold.

Alternatively to ‘crosses/crossing a/the (adjusted) sensing threshold’, other detection mechanisms like zero crossing, slope detection, or peak detection may be used for detecting a respiration of the patient.

Furthermore, the implantable medical device comprises means for determining the respiration rate of the patient by calculating a time interval between the signal crossing the sensing threshold and the signal crossing the adjusted sensing threshold.

The present invention moreover provides a computer program with program code to perform the method of the invention when the computer program is executed on a computer.

In addition, the present invention provides a computer-readable data carrier containing program code of a computer program for performing the method of the invention when the computer program is executed on a computer.

An idea of the present invention is to provide an implantable medical device, in particular an implantable cardiac monitor (ICM) that can measure respiration rate periodically and monitor changes in the rate over a long period.

This can be a very useful diagnostic tool and can be an integral part of patient's health management. Long term monitoring of respiration rate proves useful as part of vital signs monitoring. An algorithm is designed to measure the respiration rate and the resulting output can be applied to e.g. prediction of adverse cardiac events, pneumonia, and clinical deterioration.

Furthermore, calculation of the respiration rate in the implant allows a very low data rate to be used to transmit this data, rather than transmitting an entire ECG snapshot/strip. This vital sign could be more easily integrated into an electronic health record when the value/parameter is already known and does not need to be extracted.

The implantable medical device may be formed by a purely therapeutic implant. An example of a purely diagnostic implant is, e.g. a cardiac rhythm monitor. The diagnostic function consists of continuous recording of the patient's ECG and automatic evaluation of abnormalities of the heart rhythm. If such are detected, an ECG recording is stored and typically automatically transmitted to a remote monitoring system.

According to the present invention an impedance and/or accelerometer signal captured by an implantable medical device is used to detect a respiration rate of the patient. The impedance of the electrodes is a method of measuring resistance encountered by electricity passing through wires, electrodes and biological tissue. It is calculated as the ratio of the effective voltage applied to a particular circuit and the actual amount of electrical power intensity absorbed by the circuit.

According to an aspect of the invention, based on a continuous determination of the respiration rate of the patient, a mean respiration rate, a minimum respiration rate, a maximum respiration rate, change of respiration rate from baseline, confidence limits and/or a variation of the respiration rate of the patient over time is calculated. This can advantageously be used as an indicator of health status.

Moreover, a trend of a variation of the respiration rate on a day-to-day basis can be observed. The day-to-day measurements are sent to the service center of the health care provider. Any alerts in relation to this data can then be set in the service center of the health care provider, i.e. what would trigger an alert how this data will be presented to a physician.

According to a further aspect of the invention, the mean respiration rate, the minimum respiration rate, the maximum respiration rate and/or the variation of the respiration rate is transmitted to a service center of a health care provider at predefined time intervals. Further data analysis based on the transmitted data can thus be performed at the back-end, i.e. at the service center of a health care provider.

According to a further aspect of the invention, an (e.g. unfiltered) impedance and/or accelerometer signal captured by the implantable medical device, (with) at least one marker of a sense event, in particular a detected respiration of the patient is transmitted to a service center of a health care provider. The provision of said further data enables post processing, confirmation of the markers and/or additional diagnostics on the data.

According to a further aspect of the invention, an impedance and/or accelerometer signal captured by the implantable medical device, (with) at least one marker of a noise event, in particular a detected signal noise, is transmitted to a service center of a health care provider. This may indicate change in respiration status, which may help in diagnosis of sleep disorder.

According to a further aspect of the invention, upon detection of the amplitude of the impedance and/or accelerometer signal during the peak detection window, a peak value is stored in a threshold reference register. During this window, the peak of the signal is tracked. Once another peak is reached, the threshold for the following detection can be adjusted.

According to a further aspect of the invention, if no sense event is detected for a predetermined time period, a sense time out is generated, and wherein if no sense event is detected after the sense time out, the threshold reference register is reset to a predetermined value and/or the sense time out is extended to a predetermined value. The sense time out allows to wait for a certain period before it is determined that no signal is present and a new sense time out is set to an absolute minimum value for the threshold.

According to a further aspect of the invention, when the impedance and/or accelerometer signal crosses the sensing threshold, a sense event or a noise event is generated, wherein the detected respiration and/or further respiration is discarded if a sense event is followed by a noise event. This way detection accuracy can advantageously be improved.

According to a further aspect of the invention, the detection hold-off period is set in a range between 150 ms to 30000 ms, preferably between 2000 ms to 2500 ms, and in particular 2300 ms to cover a respiratory duty cycle. The detection hold-off period is chosen to accommodate a maximum measurable respiration rate of 25 breaths per minute.

According to a further aspect of the invention, the peak detection window is set in a range between 150 ms to 30000 ms, preferably between 2000 ms to 2500 ms, and in particular 2200 ms in order to identify the peak value of the impedance and/or accelerometer signal within a respiratory duty cycle. Such a peak window thus helps with correctly identifying the peak value within the respiratory cycle.

According to a further aspect of the invention, the impedance and/or accelerometer signal is half-wave rectified before respiration detection of the patient is initiated. Half-wave rectification allows only a positive signal to pass through. Half-wave rectification allows for a sensing algorithm to detect one event per respiratory cycle, as the sensing algorithm evaluates the signal with absolute values.

Alternatively, the impedance and/or accelerometer signal may be full-wave rectified before respiration detection of the patient is initiated. Full-wave rectification covers the scenario for extracting inspiration and expiration widths.

According to a further aspect of the invention, the accelerometer signal represents a movement of a chest wall or of the body of the patient, wherein (e.g. positional) changes during a breathing cycle are detected as acceleration. Using the accelerometer signal thus advantageously is an additional metric for determining the respiration rate of the patient.

According to a further aspect of the invention, respiration detection is performed using the signal having the highest amplitude, the highest performance and/or the best signal to noise ratio from the impedance or the accelerometer signal. This way a stable, continuous and accurate determination of the breath rate of the patient can be accomplished.

The herein described features of the computer-implemented method for determining a respiration rate of a patient are also disclosed for the implantable medical device, in particular implantable cardiac monitor, for determining a respiration rate of a patient and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer-implemented method for determining a respiration rate of a patient are also disclosed for the implantable medical device according to a preferred embodiment of the invention; and

FIG. 2 shows a diagram of an implantable medical device, in particular implantable cardiac monitor, for determining a respiration rate of a patient according to the preferred embodiment of the invention.

### DETAILED DESCRIPTION

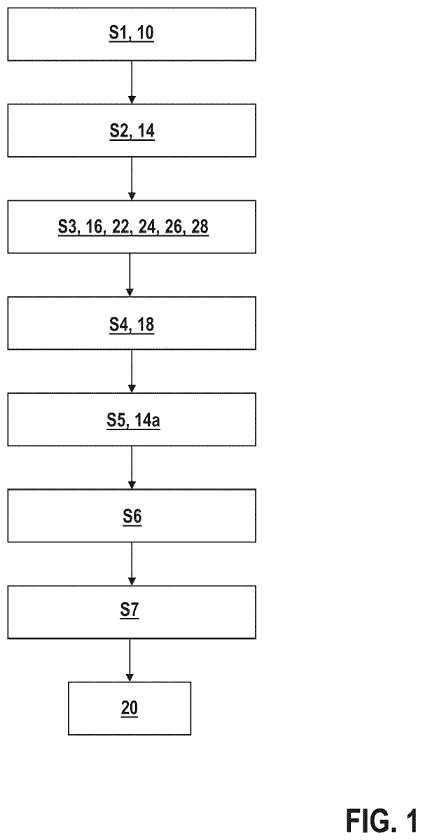

The computer-implemented method of FIG. 1 for determining a respiration rate of a patient comprises providing S1 an impedance and/or accelerometer signal 10 captured by an implantable medical device 12 and detecting S2 a respiration of the patient once the impedance and/or accelerometer signal 10 crosses a sensing threshold 14.

The method furthermore comprises starting S3 a detection hold-off period 16, starting S4 a peak detection window 18 during which, an amplitude of the impedance and/or accelerometer signal 10 is tracked, after expiry of the detection hold-off period 16, setting S5 an adjusted sensing threshold 14a based on a measured signal peak and detecting S6 a further respiration of the patient once the impedance and/or accelerometer signal 10 crosses the adjusted sensing threshold 14a.

In addition, the method comprises determining S7 the respiration rate of the patient by calculating a time interval between the signal crossing the sensing threshold 14 and the signal crossing the adjusted sensing threshold 14a.

Based on a continuous determination of the respiration rate of the patient, a mean respiration rate, a minimum respiration rate, a maximum respiration rate, change of respiration rate from baseline, at least one confidence limit and/or a variation of the respiration rate of the patient over time is calculated. The mean respiration rate, the minimum respiration rate, the maximum respiration rate and/or the variation of the respiration rate is transmitted to a service center 20 of a health care provider at predefined time intervals.

An (e.g. unfiltered) impedance and/or accelerometer signal 10 captured by the implantable medical device 12, at least one marker of a sense event 22, in particular a detected respiration of the patient, and/or at least one marker of a noise event 24, in particular detected signal noise, is transmitted to a service center 20 of a health care provider.

Upon detection of the amplitude of the impedance and/or accelerometer signal 10 during the peak detection window 18, a peak value is stored in a threshold reference register 26. If no sense event 22 is detected for a predetermined time period, a sense time out 28 is generated, and wherein if no sense event 22 is detected after the sense time out 28, the threshold reference register 26 is reset to a predetermined value and sense time out 28 is extended to predetermined value, in particular 6000 ms.

In addition, a lower absolute threshold is the lower bound limit for a target threshold and an active sensing threshold. A well above the noise-floor lower absolute threshold is required to obtain proper sensing performance. The respiration signal has small amplitude. A lower absolute threshold of 0 or 1 is required to detect the respiration rate for signals with very small amplitude. When the impedance and/or accelerometer signal 10 crosses the sensing threshold 14, a sense event 22 or a noise event 24 is generated, wherein the detected respiration and/or further respiration is discarded if a sense event 22 is followed by a noise event 24.

The detection hold-off period 16 is set in a range between 2000 ms to 2500 ms, in particular 2300 ms to cover a respiratory duty cycle. The peak detection window 18 is set in a range between 2000 ms to 2500 ms, in particular 2200 ms in order to identify the peak value of the impedance and/or accelerometer signal 10 within a respiratory duty cycle.

The impedance and/or accelerometer signal 10 is further half-wave rectified before respiration detection of the patient is initiated.

The accelerometer signal 10 represents a movement of a chest wall or of the body of the patient, wherein (e.g. positional) changes during a breathing cycle are detected as acceleration. Respiration detection is performed using the signal having the highest amplitude, the highest performance and/or the best signal to noise ratio from the impedance or the accelerometer signal 10.

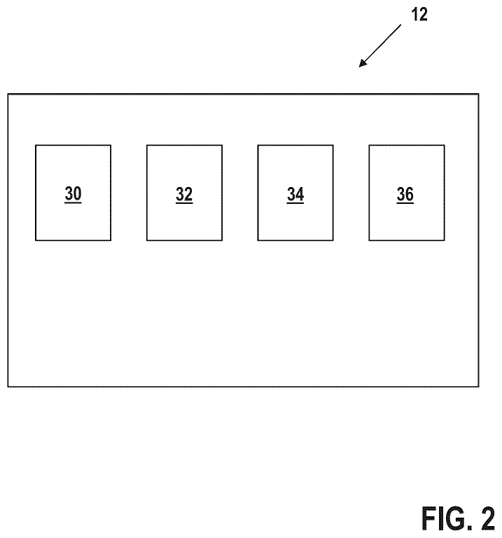

FIG. 2 shows an implantable medical device 12, in particular implantable cardiac monitor, for determining a respiration rate of a patient according to the preferred embodiment of the invention. The implantable medical device 12 comprises means 30 for capturing an impedance and/or accelerometer signal 10 and means 32 for filtering the captured impedance and/or accelerometer signal 10.

Furthermore, the implantable medical device 12 comprises a control unit 34 for detecting a respiration of the patient once the (e.g. filtered) impedance and/or accelerometer signal 10 crosses a sensing threshold 14, said control unit 34 being configured to start a detection hold-off period 16, start a peak detection window 18 during which, an amplitude of the impedance and/or accelerometer signal 10 is tracked and after expiry of the detection hold-off period 16, set an adjusted, second sensing threshold 14 based on a measured signal peak, and detect a further respiration of the patient once the impedance and/or accelerometer signal 10 crosses the adjusted, sensing threshold 14.

The implantable medical device 12 further comprises means 36 for determining the respiration rate of the patient by calculating a time interval between the signal crossing the sensing threshold 14 and the signal crossing the adjusted sensing threshold 14.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

Reference Signs

| | |
|---|---|
| 10 | impedance and/or accelerometer signal |
| 12 | implantable medical device |
| 14 | sensing threshold |
| 14a | adjusted sensing threshold |
| 16 | detection hold-off period |
| 18 | peak detection window |
| 20 | service center |
| 22 | sense event |
| 24 | noise event |
| 26 | threshold reference register |
| 28 | sense time out |
| 30 | means for capturing |
| 32 | means for filtering |
| 34 | control unit |
| 36 | means for determining |
| S1-S7 | method steps |

1. Computer-implemented method for determining a respiration rate of a patient comprising the steps of:
providing an impedance and/or accelerometer signal captured by an implantable medical device;
detecting a respiration of the patient once the impedance and/or accelerometer signal crosses a sensing threshold;
starting a detection hold-off period;
starting a peak detection window during which, an amplitude of the impedance and/or accelerometer signal is tracked;
after expiry of the detection hold-off period, setting an adjusted sensing threshold based on a measured signal peak;
detecting a further respiration of the patient once the impedance and/or accelerometer signal crosses the adjusted sensing threshold; and
determining the respiration rate of the patient by calculating a time interval between the signal crossing the sensing threshold and the signal crossing the adjusted sensing threshold.

2. Computer-implemented method of claim 1, wherein based on a continuous determination of the respiration rate of the patient, a mean respiration rate, a minimum respiration rate, a maximum respiration rate, change of respiration rate from baseline, confidence limits and/or a variation of the respiration rate of the patient over time is calculated.

3. Computer-implemented method of claim 2, wherein, the mean respiration rate, the minimum respiration rate, the maximum respiration rate and/or the variation of the respiration rate is transmitted to a service center of a health care provider at predefined time intervals.

4. Computer-implemented method of claim 1, wherein an impedance and/or accelerometer signal captured by the implantable medical device, at least one marker of a sense event, in particular a detected respiration of the patient, and/or at least one marker of a noise event, in particular detected signal noise, is transmitted to a service center of a health care provider.

5. Computer-implemented method of claim 1, wherein upon detection of the amplitude of the impedance and/or accelerometer signal during the peak detection window, a peak value is stored in a threshold reference register.

6. Computer-implemented method of claim 5, wherein if no sense event is detected for a predetermined time period, a sense time out is generated, and wherein if no sense event is detected after the sense time out, the threshold reference register is reset to a predetermined value and/or the sense time out is extended to a predetermined value.

7. Computer-implemented method of claim 1, wherein when the impedance and/or accelerometer signal crosses the sensing threshold, a sense event or a noise event is generated, wherein the detected respiration and/or further respiration is discarded if a sense event is followed by a noise event.

8. Computer-implemented method of claim 1, wherein the detection hold-off period is set in a range between 150 ms to 30000 ms, preferably between 2000 ms to 2500 ms, and in particular 2300 ms to cover a respiratory duty cycle.

9. Computer-implemented method of claim 1, wherein the peak detection window is set in a range between 150 ms to 30000 ms, preferably between 2000 ms to 2500 ms, and in particular 2200 ms in order to identify the peak value of the impedance and/or accelerometer signal within a respiratory duty cycle.

10. Computer-implemented method of claim 1, wherein the impedance and/or accelerometer signal is half-wave rectified before respiration detection of the patient is initiated.

11. Computer-implemented method of claim 1, wherein the accelerometer signal represents a movement of a chest wall or of the body of the patient, wherein changes during a breathing cycle are detected as acceleration.

12. Computer-implemented method of claim 1, wherein respiration detection is performed using the signal having the highest amplitude, the highest performance and/or the best signal to noise ratio from the impedance or the accelerometer signal.

13. Implantable medical device, in particular implantable cardiac monitor, for determining a respiration rate of a patient, comprising:
means for capturing an impedance and/or accelerometer signal;
means for filtering the captured impedance and/or accelerometer signal;
a control unit for detecting a respiration of the patient once the filtered impedance and/or accelerometer signal crosses a sensing threshold, said control unit being configured to start a detection hold-off period, start a peak detection window during which, an amplitude of the filtered impedance and/or accelerometer signal is tracked,
after expiry of the detection hold-off period, set an adjusted, second sensing threshold based on a measured signal peak, and detect a further respiration of the patient once the filtered impedance and/or accelerometer signal crosses the adjusted sensing threshold, and
means for determining the respiration rate of the patient by calculating a time interval between the signal crossing the sensing threshold and the signal crossing the adjusted sensing threshold.

14. Computer program with program code to perform the method of claim 1 when the computer program is executed on a computer.

15. Computer-readable data carrier containing program code of a computer program for performing the method of claim 1 when the computer program is executed on a computer.
